SEQUENCE LISTING (10 sequences)

SEQ ID NO: 1 - protein, 8 aa, Artificial Sequence
  P8GB#1 which is a peptide having superior binding affinity for graphitic material
DSWAADIP

SEQ ID NO: 2 - protein, 8 aa, Artificial Sequence
  P8GB#5 which is a peptide having superior binding affinity for graphitic material
DNPIQAVP

SEQ ID NO: 3 - DNA, 7222 bp, Artificial Sequence
  cloning vector M13KE
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagtggtacctttctattctcactcggccgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgttccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 4 - DNA, 41 bp, Artificial Sequence
  BamH I_SM_upper which is a primer used for site-directed mutation of M13KE vector
aaggccgcttttgcgggatcctcaccctcagcagcgaaaga

SEQ ID NO: 5 - DNA, 41 bp, Artificial Sequence
  BamH I_SM_lower which is a primer used for site-directed mutation of M13KE vector
tctttcgctgctgagggtgaggatcccgcaaaagcggcctt

SEQ ID NO: 6 - DNA, 90 bp, Artificial Sequence
  M13HK_P8_primer which is an extension primer used for preparation of the phage display p8 peptide library
ttaatggaaacttcctcatgaaaaagtctttagtcctcaaagcctctgtagccgttgctaccctcgttccgatgctgtctttcgctgctg

SEQ ID NO: 7 - DNA, 95 bp, Artificial Sequence
  M13HK_P8 which is a library oligonucleotide used for preparation of the phage display p8 peptide library
aaggccgcttttgcgggatccnnmnnmnnmnnmnnmnnmnnmncagcagcgaaagacagcatcggaacgagggtagcaacggctacagaggcttt

SEQ ID NO: 8 - protein, 8 aa, Artificial Sequence
  P8GB#3 which is a peptide screened by biopanning method
DTKWTGGE

SEQ ID NO: 9 - protein, 8 aa, Artificial Sequence
  P8GB#6 which is a peptide screened by biopanning method
VTAVPNDT

SEQ ID NO: 10 - protein, 3 aa, Artificial Sequence
  P8GB#8 which is a peptide as a negative control
EGE

(19) United States
(12) Patent Application Publication
(10) Pub. No.: US 2014/0309126 A1
(43) Pub. Date: Oct. 16, 2014

(54) PEPTIDE BINDING TO GRAPHITIC MATERIALS AND PHAGE INCLUDING SAME

(71) Applicant: Korea Institute of Science and Technology, Seoul (KR)

(72) Inventors: Hyunjung YI, Seoul (KR); Ki Young LEE, Seoul (KR); Chaun JANG, Busan (KR); Joonyeon CHANG, Seoul (KR)

(73) Assignee: Korea Institute of Science and Technology, Seoul (KR)

(21) Appl. No.: 14/171,898

(22) Filed: Feb. 4, 2014

(30) Foreign Application Priority Data
Apr. 15, 2013 (KR) 10-2013-0041173

(51) Int. Cl. C07K 7/06

(52) U.S. Cl. CPC C07K 7/06
USPC 506/9; 530/328; 435/235.1; 435/176

(57) ABSTRACT

The present disclosure provides a peptide including one or more amino acid sequence selected from a group consisting of SEQ ID NO 1 and SEQ ID NO 2 and binding specifically to a graphitic material, a phage including same, and a graphitic material including a graphitic surface on which the peptide or the phage is arranged.

### CROSS-REFERENCE TO RELATED APPLICATION

This application claims priority to Korean Patent Application No. 10-2013-0041173, filed on Apr. 15, 2013, and all the benefits accruing therefrom under 35 U.S.C. §119, the contents of which in its entirety are herein incorporated by reference.

### BACKGROUND

1. Field

The present disclosure relates to a novel peptide specifically binding to graphitic materials.

2. Description of the Related Art

Recently, researches are actively carried out on utilization of the superior electrical, thermal, optical and mechanical properties of low-dimensional carbon materials such as graphene, carbon nanotube, etc. in various applications.

In general, permanent modification of the surface of nanocarbon materials through chemical reactions is employed for modification of their properties. However, if the surface is permanently modified through chemical reaction, the intrinsic properties of the low-dimensional carbon materials such as high electrical conductivity are greatly deteriorated.

Accordingly, there is an increasing need of a novel method capable of providing various functionalities without disrupting the superior properties of nanocarbon materials.

In this regard, molecular recognition is a method of binding to a desired substance without chemical reaction utilizing the selectivity of a biomaterial and can be found in nature, for example, in the binding between complementary DNA sequences, antigen-antibody interaction, etc. Recently, researches are actively conducted on modification of the surface of nanocarbon materials with minimized deterioration of their properties using peptides that specifically bind to the nanocarbon materials through molecular recognition.

Most of the current researches on functionalization of carbon nanotube, etc. using peptides are based on the commercially available p3 peptide library (Fabricating genetically engineered high-power lithium-ion batteries using multiple virus genes, Yun Jung Lee et al., Science Vol. 324, 2009. 05. 22.). However, examples of the functionalization of a nanocarbon material using actually discovered peptides are already reported in a biosensor, but their applications are also very limited. It is because a synergic effect between the peptides cannot be expected since, although a large quantity of peptides are needed to functionalize the nanocarbon material, the peptides are small in size. In addition, since the peptides derived from the p3 peptide library are present at small copy number of about 5 on the tip of phage particles, it is difficulty to functionalize the nanocarbon material using the phages to which the peptides are bound.

### REFERENCES OF THE RELATED ART

### Non-Patent Document

Fabricating genetically engineered high-power lithium-ion batteries using multiple virus genes (Yun Jung Lee et al., Science Vol. 324, 2009. 05. 22.)

### SUMMARY

The present disclosure is directed to providing a peptide having superior binding affinity for a graphitic material and allowing functionalization of a graphitic material having a graphitic surface using a phage comprising the peptide.

In one aspect, there is provided a peptide comprising one or more amino acid sequence selected from a group consisting of SEQ ID NO 1 and SEQ ID NO 2, a phage comprising same, and a graphitic material having a graphitic surface on which the peptide or phage is arranged.

In another aspect, there is provided a method for screening the peptide, comprising:

(1) preparing a phage display p8 peptide library;

(2) conjugating the phage display p8 peptide library onto a surface of a graphitic material; and

(3) screening a peptide that specifically binds to the surface of the graphitic material from the phage display p8 peptide library by removing peptides that are not bound to the surface of the graphitic material.

The peptide according to the present disclosure binds specifically to a graphitic surface with higher binding affinity than the existing peptides. Also, if the peptide according to the present disclosure is comprised in a phage, it can be easily amplified using the phage because the peptide has a very large copy number, differently from the other peptides and a large amount of the peptide can be comprised to the phage. Accordingly, no additional protein purification process is required and the cost of peptide preparation can be saved greatly.

In addition, not only the peptide according to the present disclosure but also the phage comprising the peptide may be used to functionalize a material having a graphitic surface. That is to say, by utilizing the strong binding affinity of the peptide included in the phage for the graphitic surface as well as the relatively large size of the phage than the peptide, the phage may be arranged on the graphitic surface with directionality to form a system. In this manner, the properties of the material having the graphitic surface may be tuned by controlling the direction or type of the arrangement.

Also, the strong binding affinity of the peptide for the graphitic surface may be utilized in various applications, including energy storage devices such as lithium-ion batteries, solar cells, supercapacitors, etc., displays, biosensors, or the like.

### BRIEF DESCRIPTION OF THE DRAWINGS

The above and other aspects, features and advantages of the disclosed exemplary embodiments will be more apparent from the following detailed description taken in conjunction with the accompanying drawings in which:

FIGS. 1 and 2 show the DNA sequence of an M13KE vector according to an exemplary embodiment of the present disclosure;

FIG. 3 schematically describes a biopanning method for screening peptide sequences that bind to a graphitic surface;

FIG. 4 schematically shows the structure of an M13 phage according to an exemplary embodiment of the present disclosure;

FIG. 5 shows atomic force microscopic images obtained after binding the phages comprising the peptide of the present disclosure to the surface of graphene;

FIG. 6 shows an experimental result of comparing the binding affinity for a graphitic surface depending on the amino acid sequence of peptides;

FIG. 7 shows the relationship between the amino acid sequence of each peptide and its hydrophobic property; and

FIG. 8 shows an atomic force microscopic image obtained after arranging the phages comprising the peptide of the present disclosure on the surface of graphene.

### DETAILED DESCRIPTION

As used herein, the term “graphitic material” refers to a material having a graphitic surface, i.e. a surface on which carbon atoms are arranged in hexagonal shape. Any material having a graphitic surface is included, regardless of physical, chemical or structural properties.

Exemplary embodiments now will be described more fully hereinafter with reference to the accompanying drawings, in which exemplary embodiments are shown.

In one aspect, the present disclosure provides a peptide comprising one or more amino acid sequence selected from a group consisting of SEQ ID NO 1 and SEQ ID NO 2. The amino acid sequences of SEQ ID NOS 1 and 2 are as follows.

(SEQ ID NO 1)
DSWAADIP
(SEQ ID NO 2)
DNPIQAVP

In an exemplary embodiment of the present disclosure, the peptide including one or more amino acid sequence selected from SEQ ID NOS 1 and 2 can selectively and specifically bind to (conjugate with) a graphitic material. The graphitic material is not particularly limited as long as it is a material having a surface on which carbon atoms are arranged in hexagonal shape such as, for example, graphite, graphene, highly oriented pyrolytic graphite (HOPG), carbon nanotube, fullerene, etc. For example, as one of the graphitic materials, graphene is a nanomaterial consisting of carbon, having a very small thickness of about 0.3 nm as well as superior conductivity and physical and chemical stability. In an exemplary embodiment of the present disclosure, the peptide may strongly bind to the surface of graphene selectively and specifically. Through this, functionality may be provided to the surface of graphene without negatively affecting the properties of graphene. Further, since the peptide has very high binding affinity, it can solve the problem of aggregation or dissolution of the existing peptide having low binding affinity.

In an exemplary embodiment of the present disclosure, the peptide may be derived from a phage display p8 peptide library, e.g. a filamentous phage. Specifically, it may be obtained from an M13 phage display p8 peptide library.

In another aspect, the present disclosure provides a method for screening a peptide from the M13 phage display p8 peptide library, comprising:

(1) preparing an M13 phage display p8 peptide library;

(2) conjugating the M13 phage display p8 peptide library onto a surface of a graphitic material; and

(3) screening a peptide that specifically bind to the surface of the graphitic material from the M13 phage display p8 peptide library by removing peptides that are not bound to the surface of the graphitic material.

The phage display p8 peptide library may be prepared through site-directed mutation of a phage vector followed by insertion of the peptide to the mutant vector.

Specifically, in an exemplary embodiment of the present disclosure, the M13 phage display p8 peptide library in (1) may be prepared by a method comprising:

(a) preparing a mutant M13 phage vector through site-directed mutation of an M13 phage vector; and

(b) preparing an M13 phage display p8 peptide library from the mutant M13 phage vector using a restriction enzyme.

In an exemplary embodiment of the present disclosure, the M13 phage vector may be an M13KE vector (NEB, product #NO316S). In an exemplary embodiment of the present disclosure, when the M13KE vector is used, the 1381st base pair C of the M13KE vector is site-directedly mutated to G to prepare an M13HK vector.

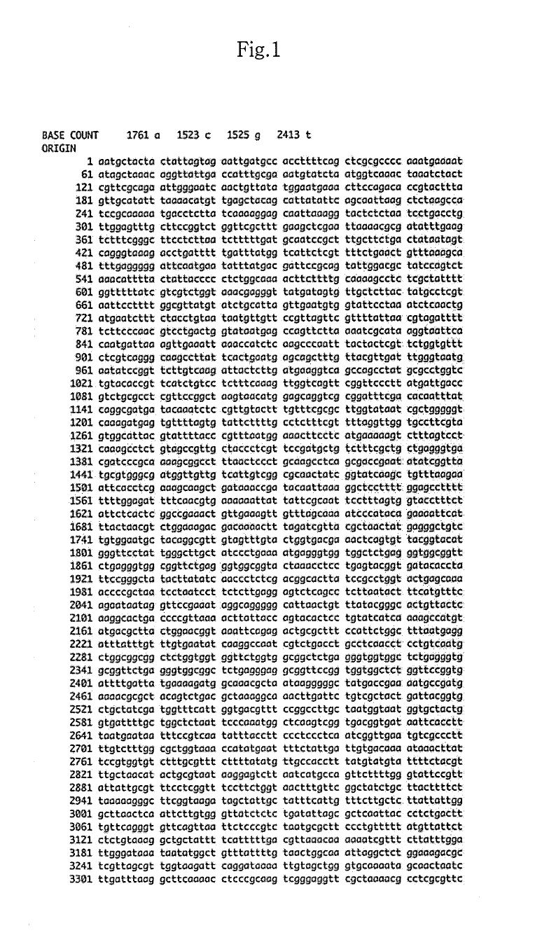

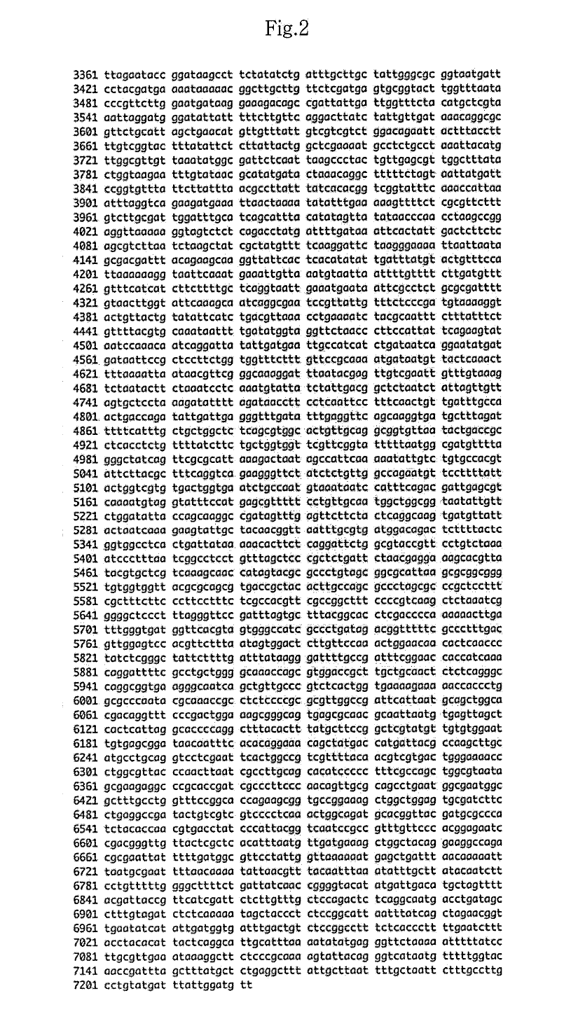

The M13KE vector (NEB, product #NO316S) is a cloning vector consisting of 7222-bp DNAs and its genetic information is available from the Internet (https://www.neb.com/˜/media/NebUs/Page%20Images/Tools%20and%20Resources/Interactive/%20Tools/DNA%20Sequences%20and%20Maps/Text%20Documents/m13kegbk.txt). The DNA sequence of the M13KE vector (SEQ ID NO 3) is shown in FIGS. 1 and 2. It can also be confirmed from the following references the contents of which in its entirety are herein incorporated by reference.
The maltose-binding protein as a scaffold for monovalent display of peptides derived from phage libraries; Zwick, M. B., Bonnycastle, L. L., Noren, K. A., Venturini, S., Leong, E., Barbas, C. F. III, Noren, C. J. and Scott, J. K.; Anal Biochem 264 (1), 87-97 (1998).
Construction of high-complexity combinatorial phage display peptide libraries; Noren, K. A. and Noren, C. J.; Methods 23 (2), 169-178 (2001).
Direct Submission; Paschal, B. M.; Submitted (19-Oct.-2007) Research Department, New England Biolabs, 240 County Road, Ipswich, Mass. 01938, USA.

As the restriction enzyme, BspHI (NEB, product #R0517S) or BamHI restriction enzyme (NEB, product #R3136T) may be used. But, any one can be used without particular limitation as long as genetic recombination is possible.

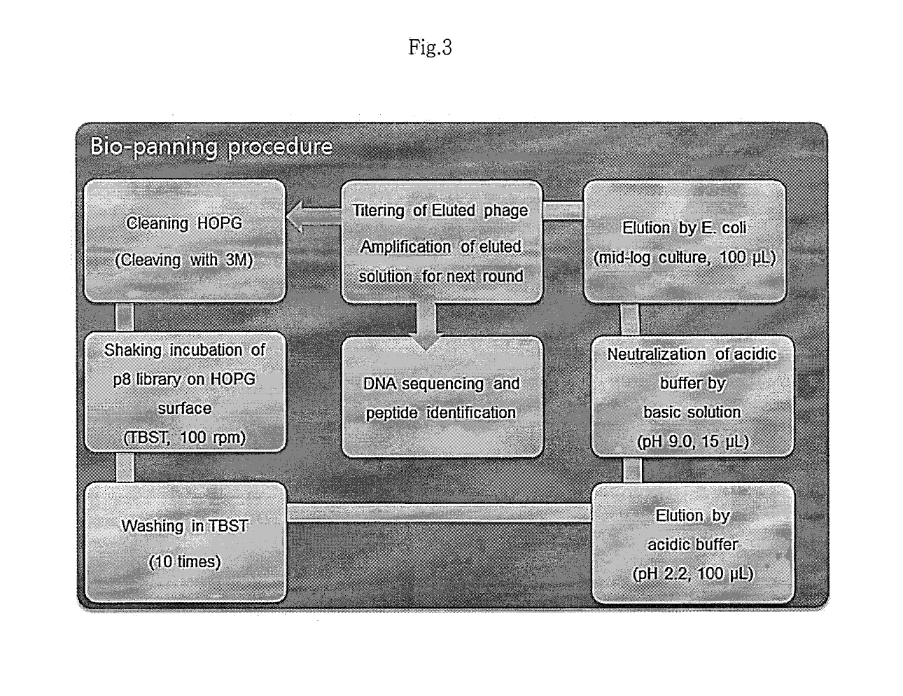

In an exemplary embodiment of the present disclosure, the conjugation of the M13 phage display p8 peptide library onto the graphitic surface in (2) is achieved by a biopanning method. Specifically, the biopanning may be performed as follows (see FIG. 3).

First, an M13 phage display p8 peptide library is prepared in a buffer and conjugated with a graphitic surface. In the past, since a carbon nanotube film surface which is vulnerable to damage was used as the graphitic surface, it was difficult to obtain a peptide with high binding affinity. In the present disclosure, to solve this problem, a substrate having a graphitic surface such as HOPG is used and the surface is detached using a tape immediately before use to minimize the defect of the sample surface due to, for example, oxidation.

After the p8 peptide library is conjugated with the graphitic surface and the solution is removed, the surface is washed with a buffer and then the washed HOPG surface is reacted with an acidic buffer to elute peptides that bind non-selectively. The phage remaining without being eluted to the acidic buffer is eluted with an E. coli culture in mid-log phase. A portion of the eluted culture is left for DNA sequencing and peptide identification and the remainder is amplified to prepare a sub-library for the next round. The above procedure is repeated using the prepared sub-library. The left plaque may be subjected to DNA sequencing to obtain the p8 peptide sequence, and the sequence may be analyzed to obtain the peptide sequence that reacts with the graphitic surface according to the present disclosure.

Although biopanning has been widely employed to select for peptides that bind with a desired material, it has never been used to screen peptides from the M13 phage display p8 peptide library prepared as described above.

The peptide screened from the phage display p8 peptide library according to an exemplary embodiment of the present disclosure has a large copy number of about 2700 copies, whereas the peptide screened from the existing phage display p3 peptide library has a copy number of about 5. Accordingly, the peptide can be easily amplified using the phage and no additional protein purification process is required. As a result, the cost of peptide preparation can be saved greatly. In addition, since the p8 peptide present on the surface of, e.g., the M13 phage, is used, the peptide can be obtained easily by amplifying the phage.

Since the peptide according to an exemplary embodiment of the present disclosure binds selectively and specifically to the graphitic surface of graphene, carbon nanotube, fullerene, etc., which are high value-added nanocarbon materials, additional functionality may be provided without harming the properties of the nanomaterial. Most importantly, the peptide screened according to the method of the present disclosure has about 10 times stronger binding affinity than that of a peptide which has not been mutated using the restriction enzyme (negative control).

In another aspect, the present disclosure provides a graphitic material including a graphitic surface on which the peptide including one or more amino acid sequence selected from a group consisting of SEQ ID NO 1 and SEQ ID NO 2 is specifically bound.

In another aspect, the present disclosure provides a phage including the peptide including one or more amino acid sequence selected from a group consisting of SEQ ID NO 1 and SEQ ID NO 2. Specifically, in an exemplary embodiment, the present disclosure provides a filamentous phage wherein the peptide including one or more amino acid sequence selected from a group consisting of SEQ ID NO 1 and SEQ ID NO 2 is displayed on a coat protein. The filamentous phage may be, for example, an M13 phage. The M13 phage may be easily engineered genetically to include the peptide for selective binding onto the graphitic surface.

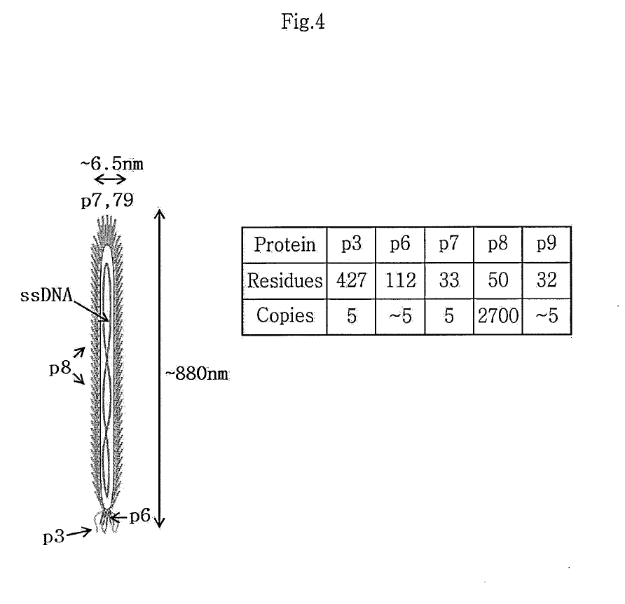

In an exemplary embodiment of the present disclosure, the coat protein of the M13 phage may be selected from a group consisting of p3, p6, p7, p8 and p9. More specifically, the peptide may be displayed on p8 (see FIG. 4). p3, p6, p7 and p9 are minor coat proteins and p8 is a major coat protein. Whereas the copy number of all the minor coat proteins is very small as 5 or smaller, the copy number of the major coat protein p8 is very large as approximately 2700. Further, since p8 is present on the body of the phage, the area where the peptide can be displayed is relatively very large. Accordingly, if the peptide of the present disclosure is displayed on the coat protein p8 which is present on the body of the M13 phage, the body of the phage itself, which is in micrometer size (height=880 nm, diameter≦6.5 nm), can be used to functionalize the nanocarbon material such as graphene.

Also, whereas binding between graphitic materials cannot be induced using the nanometer-sized peptide itself, binding between homogeneous or heterogeneous nanocarbon materials can be formed in a non-destructive manner using the micrometer-sized phage wherein the peptide is displayed on the body. Accordingly, it is possible to realize various nanocarbon materials having percolated network structures, which are critical in applications to energy conversion or storage devices, flexible electronic devices, biosensors, etc.

Further, since the filamentous phage has a thread-like structure, the filamentous phage wherein the peptide according to an exemplary embodiment of the present disclosure is displayed on the coat protein may provide strong binding affinity for binding to a graphitic surface, owing to large contact area of the peptide.

In another aspect, the present disclosure provides a graphitic material on which the phage according to the present disclosure is arranged.

The graphitic material on which the M13 phage is arranged may be, for example, graphite, graphene, highly oriented pyrolytic graphite (HOPG), carbon nanotube or fullerene.

In an exemplary embodiment, the phage according to the present disclosure itself may be arranged on the graphitic surface of the graphitic material to form a system. Accordingly, not only the high binding affinity and specificity for the graphitic surface of the peptide included in the phage but also the liquid-crystalline property of the phage itself can be utilized and, hence, a graphitic surface of large area can be functionalized.

In an exemplary embodiment of the present disclosure, the filamentous phage may be arranged on the graphitic surface with directionality using the thread-like structure of the phage itself. For example, it may be arranged in a row in a specific direction. In this case, the binding affinity of the peptide present on the body of the phage for the graphitic surface is enhanced and a wire property may be utilized. The phage arranged in a row may provide anisotropic functionality to the graphitic surface. This is distinguished from the existing peptide which can provide only isotropic or random functionalization. For example, the anisotropic functionalization may allow realization of a new-concept electronic device by providing additional specific electrical property to graphene (Anisotropic behaviours of massless Dirac fermions in graphene under periodic potentials, Cheol-Hwan Park et. al, Nature Physics 2008, vol. 4, 213-217). In another exemplary embodiment, the phage according to the present disclosure may be arranged to form a structure having specific directionality, such as a layered (e.g., smectic), nematic, spiral or lattice structure. Accordingly, various functionalities may be provided onto the graphitic surface using the various arrangement structures of the phage (Chiral Smectic C Structures of Virus-Based Films, Seung-Wuk Lee et. al, Langmuir 2003, Vol. 19, No. 5, 1592-1598). The contents of the cited literatures in its entirety are herein incorporated by reference.

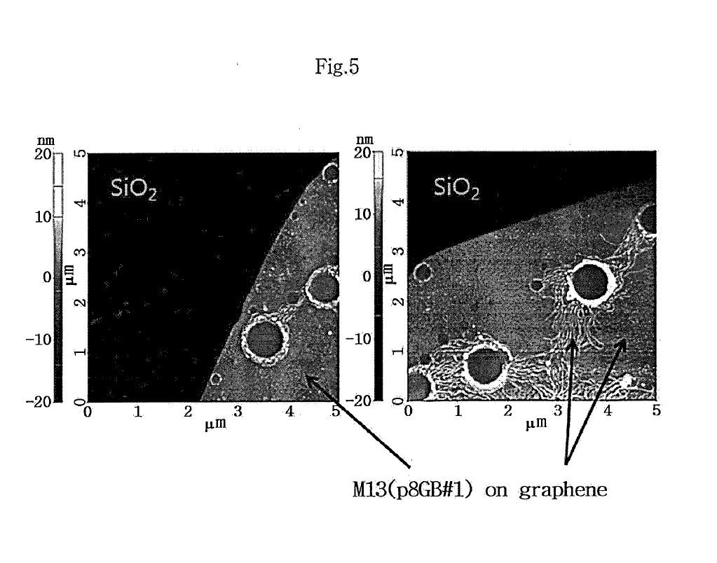

FIG. 5 shows atomic force microscopic (AFM) images obtained after binding the M13 phage including the peptide having an amino acid sequence of SEQ ID NO 1 according to the present disclosure in the coat protein p8 to the surface of graphene (kish graphite, Covalent). The graphene to which the M13 phage is bound was prepared by a dip coating method of dipping and then pulling up the graphene placed on an SiO₂ substrate in a solution of the M13 phage in ultrapure distilled water (pH 5.3) prepared to a concentration of 1.25×10¹³ viral particles/mL. The higher the pH of the solvent used in the phage solution, the stronger is the electrostatic repulsive force between the phage particles and hence the larger the spacing between the phages. Also, the spacing between the phages arranged on the graphitic surface may be narrower as the concentration of the phage in the prepared solution is higher. Accordingly, the number, spacing, etc. of the phage arranged on the graphitic surface may be controlled by adjusting the solvent pH and phage concentration.

When the surface of a nanomaterial is photographed by atomic force microscopy (AFM), the higher portion looks brighter. In FIG. 5, the SiO₂ substrate looks black with low brightness, and the graphene looks brighter. This suggests that the phage including the peptide having an amino acid sequence of SEQ ID NO 1 according to the present disclosure is not bound to the SiO₂ substrate but is bound only to the graphene surface. Accordingly, it can be seen that the peptide having an amino acid sequence of SEQ ID NO 1 has high selectivity and specificity for the graphene surface.

The large circles on the graphene surface shown in FIG. 5 are air bubbles formed on the graphene surface. It can be confirmed that the phage covers the graphene surface in one layer, except for the air bubbles. Also, it can be seen that, around the air bubbles, the phage immobilized by the air bubble is not directly bound to the graphene but is bound to the phage bound to the graphene, thereby forming a double layer. In particular, referring to the right-side image in FIG. 5, since the phages around the air bubbles repel one another, contact is minimized and the individual phages are clearly seen. In contrast, the phages bound to the graphene are flattened to maximize the binding area because of the strong binding affinity between the peptide included in the phage and the graphene. That is to say, the individual phages are not clearly seen but the phages are seen to be arranged on the graphitic surface as one structure, thus forming a functionalized system.

As such, the peptide according to the present disclosure has selective binding affinity for the graphitic surface such as graphene and also has strong binding affinity enough to change the morphology of the phage itself.

In an exemplary embodiment, the phage according to the present disclosure is capable of functionalizing the graphitic surface in a non-destructive manner and is capable of functionalizing in micrometer scale as compared to when only the nanometer-sized peptide is bound to the graphitic surface. For example, the phage can be used to induce binding between homogeneous or heterogeneous carbon compounds. In addition, a conducting network may be formed by connecting nanocarbon materials. Accordingly, the phage can be used for high-performance electrodes and sensors. As described, since the present disclosure allows not only the binding of the peptide of the present disclosure to the graphitic material but also the binding of the phage on which the peptide is displayed to the graphitic material, it is expected to provide new applications of graphitic materials having graphitic surfaces.

### EXAMPLES

Hereinafter, the present disclosure will be described in detail through examples and test examples. However, the following examples and test examples are for illustrative purposes only and it will be apparent to those of ordinary skill in the art that the scope of the present disclosure is not limited by the examples and test examples.

### Example 1

### Preparation of M13 Phage Display p8 Peptide Library

An M13 phage display p8 peptide library was prepared as follows. First, the 1381st base pair C of the M13KE vector (NEB, product #N0316S) was site-directedly mutated to G to prepare an M13HK vector.

The base sequences of the oligonucleotides used for the site-directed mutation were as follows:

(SEQ ID NO 4)
5′-AAG GCC GCT TTT GCG GGA TCC TCA CCC
TCA GCA GCG AAA GA-3′,
and
(SEQ ID NO 5)
5′-TCT TTC GCT GCT GAG GGT GAG GAT CCC
GCA AAA GCG GCC TT-3′.

A phage display p8 peptide library was prepared from the prepared M13HK vector using the restriction enzymes BspHI (NEB, product #R0517S) and BamHI (NEB, product #R3136T).

The base sequences of the oligonucleotides used for the preparation of the phage display p8 peptide library were as follows:

(SEQ ID NO 6)
5′-TTA ATG GAA ACT TCC TCA TGA AAA AGT CTT
TAG TCC TCA AAG CCT CTG TAG CCG TTG CTA CCC
TCG TTC CGA TGC TGT CTT TCG CTG CTG-3′,
and
(SEQ ID NO 7)
5′-AAG GCC GCT TTT GCG GGA TCC NNM NNM NNM
NNM NNM NNM NNM NCA GCA GCG AAA GAC AGC ATC
GGA ACG AGG GTA GCA ACG GCT ACA GAG GCT TT-3′.

The base sequence of the prepared phage display p8 peptide library had a diversity of 4.8×10⁷ plaque-forming unit (PFU) and had a copy number of about 1.3×10⁵ per sequence.

### Example 2

### Screening of Peptide

Peptides were screened by a biopanning method by binding the phage display p8 peptide library prepared in Example 1 to a graphitic surface. The biopanning was conducted as follows.

First, a fresh surface was detached from highly oriented pyrolytic graphite (HOPG; SPI, product #439HP-AB) as a material having a graphitic surface using a tape immediately before use to minimize the defect of the sample surface due to, for example, oxidation.

A HOPG substrate having a relatively large grain size of 100 μm or smaller was used.

Subsequently, the phage display p8 peptide library of 4.8×10¹⁰ PFU (4.8×10⁷ diversities, 1000 copies per each sequence) prepared in Example 1 was prepared in 100 μL of Tris-buffered saline (TBS) and conjugated with the HOPG surface for 1 hour in a shaking incubator at 100 rpm. 1 hour later, the solution was removed and the HOPG surface was washed 10 times in TBS. The washed HOPG surface was reacted with pH 2.2 Tris-HCl as an acidic buffer for 8 minutes to remove (elute) peptides that reacted non-selectively, and the remaining phage was eluted with an XL-1 blue E. coli culture in mid-log phase for 30 minutes. A portion of the eluted culture was left for DNA sequencing and peptide identification and the remainder was amplified to prepare a sub-library for the next round. The above procedure was repeated using the prepared sub-library. Meanwhile, the left plaque was subjected to DNA sequencing to obtain the p8 peptide sequence, and the sequence was analyzed to obtain the peptide sequence that reacts with the graphitic surface.

Table 1 shows some amino acid sequences of the peptides screened by the biopanning method.

TABLE 1

| SEQ ID NO | Amino acid sequence |
|---|---|
| p8GB #1 (SEQ ID NO 1) | DSWAADIP |
| p8GB #3 (SEQ ID NO 8) | DTKWTGGE |
| p8GB #5 (SEQ ID NO 2) | DNPIQAVP |
| p8GB #6 (SEQ ID NO 9) | VTAVPNDT |
| p8GB #8 (M13HK, negative | EGE |
| control, SEQ ID NO 10) | |

### Test Example 1

### Comparison of Binding Affinity

The following experiment was conducted to compare the binding affinity of the peptide sequences p8 GB #1, 3, 5, 6 and 8 screened in Example 2 for the graphitic surface.

First, M13 phages each including the peptide sequences p8 GB #1, 3, 5, 6 and 8 were prepared according to the biopanning method used in Example 2 and, after conjugating them with HOPG under the same conditions, binding affinity was compared by counting the number of phages remaining after washing.

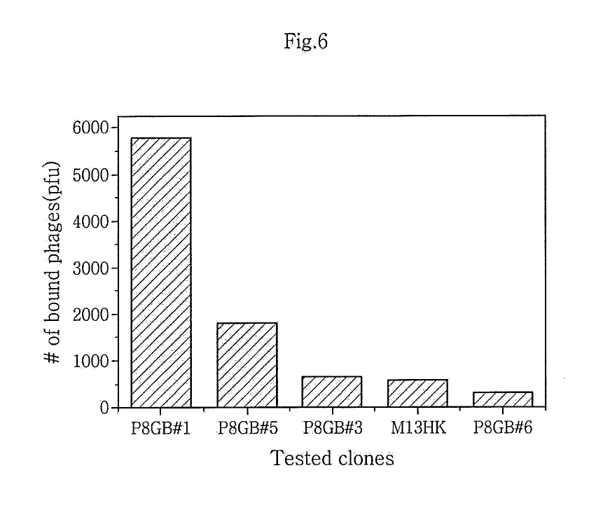

That is to say, each of the five peptide sequences was prepared in 100 μL of Tris-buffered saline (TBS) and conjugated with the HOPG surface for 1 hour in a shaking incubator at 100 rpm. 1 hour later, the solution was removed and the HOPG surface was washed 10 times in TBS. The washed HOPG surface was reacted with pH 2.2 Tris-HCl as an acidic buffer for 8 minutes to remove (elute) peptides that reacted non-selectively, and the remaining phage was eluted with an XL-1 blue E. coli culture in mid-log phase for 30 minutes. The number of the phages in the eluted culture was counted by tittering. The result is shown in FIG. 6.

As seen from FIG. 6, p8GB #1 (SEQ ID NO 1) according to an exemplary embodiment of the present disclosure showed about 9.6 times stronger binding affinity and p8 GB #5 (SEQ ID NO 2) according to another exemplary embodiment of the present disclosure showed about 2.9 times stronger binding affinity as compared to p8 GB #8 (M13HK, negative control). In contrast, p8 GB #3 showed only about 1.1 times stronger binding affinity as compared to p8 GB #8 (negative control).

Accordingly, it was confirmed that the peptide according to the present disclosure has very higher binding affinity as compared to peptides of other sequences derived from the same M13 phage display p8 peptide library.

### Test Example 2

### Analysis of Hydrophobic Property of Peptide Sequences

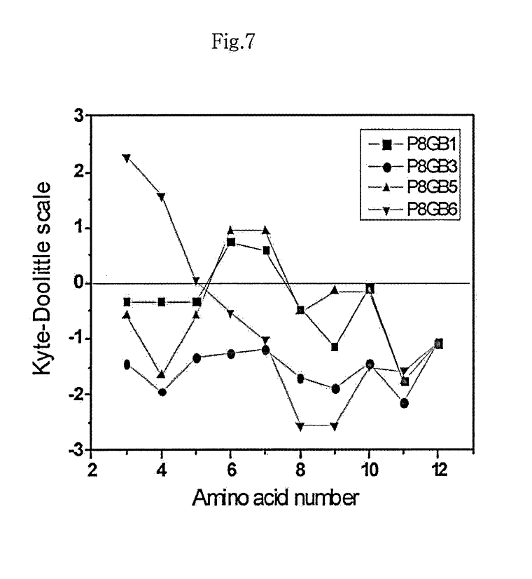

In order to investigate why the peptide sequences p8 GB #1 and #5 according to the present disclosure have significantly higher binding affinity than p8 GB #3 and #6, the hydrophobic property of each peptide sequence was analyzed according to the Kyte-Doolittle scale (window size=5), and the result is shown in FIG. 7. In FIG. 7, the more positive (+) value in the ordinate means stronger hydrophobicity, and the more negative (−) value means stronger hydrophilicity.

As seen from FIG. 7, both the peptide sequences p8 GB #1 and p8 GB #5 according to the present disclosure showed high hydrophobicity of 0.6 or higher in the 5th to 6th amino acid sequences. The reason why p8 GB #1 exhibits higher binding affinity than p8 GB #5 may be due to the presence of the aromatic tryptophan (W) residue in p8 GB #1. That is to say, it is though that the presence of the aromatic residue having good reactivity with the graphitic surface in the middle of the amino acid sequence of p8 GB #1 leads to high binding affinity.

In contrast, it is though that p8 GB #3 and p8 GB #6 exhibit low binding affinity for the graphitic surface because they show low hydrophobicity in the 5th to 6th amino acid sequences. Although p8 GB #3 also has the aromatic tryptophan residue, p8 GB #3 has low hydrophobicity in the 5th to 6th amino acid sequences, and the hydrophobic property has a stronger effect on the binding affinity than the presence of the aromatic residue.

In case of p8 GB #6, a portion of the sequence has hydrophobic property but the amino acids in the 5th and 6th positions are hydrophilic. As a result, it exhibits even lower binding affinity than the negative control p8 GB #8 (EGE). This may be because the inserted peptide p8 GB #6 inhibits even the non-specific binding of the phage.

Accordingly, it can be seen that a peptide having an aromatic residue does not always bind strongly to the graphitic surface, and hydrophobic property, particularly its pattern, is important.

### Test Example 3

### Preparation of Graphene on which M13 Phage Wherein Peptide is Displayed

Graphene on which the M13 phage wherein the peptide having an amino acid sequence of SEQ ID NO 1 according to the present disclosure is arranged was prepared as follows.

First, an M13 phage including the peptide sequence p8 GB #1 (SEQ ID NO 1) was prepared using the biopanning method used in Example 2. Considering that the spacing between phages may increase at higher pH because of increased electrostatic repulsion between the phage particles, a phage solution having a concentration of 1×10¹³ viral particles/mL was prepared using ultrapure distilled water adjusted to pH 7.0. The concentration of the phage solution can be calculated by Equation 1.

Phage concentration(viral particles/mL)=1.6×10¹⁶×O.D._{viral solution}/7237 Equation 1

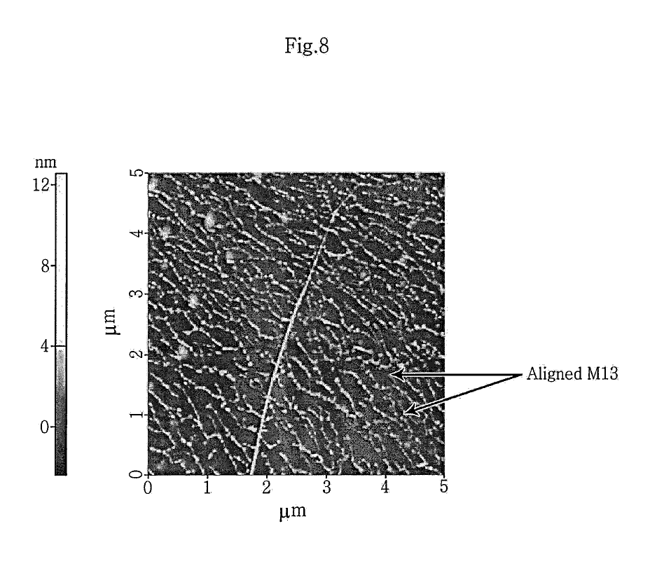

Then, graphene (kish graphite, Covalent) was placed on an SiO₂/Si substrate (EPI-Prime Si wafer with 300 nm dry oxidized SiO₂, Siltron, Inc., Korea) using the taping method. The substrate with the graphene placed thereon was dipped in the solution of the phage on which the p8 GB #1 peptide is displayed prepared above, and then pulled up at a rate of 10 μm/min out of the solution (dip coating). The arrangement of the peptide on the surface of the substrate was observed by atomic force microscopy (AFM). The result is shown in FIG. 8.

Referring to FIG. 8, it can be seen that the phage is aligned well in a predetermined direction on the graphene in spite of the morphological change of the phage due to the binding to the graphene. Specifically, because the peptide having an amino acid sequence of SEQ ID NO 1 is included in the body coat protein, p8, of the M13 phage, the phage is arranged in a thread-like shape owing to strong binding affinity of the peptide for the graphene surface. In particular, it can be seen that the thread-like phages are aligned in a row in the same direction. This means that the phage according to the present disclosure may be used to anisotropically functionalize a material having a graphitic surface by arranging the phage on the graphitic surface with directionality.

While the exemplary embodiments have been shown and described, it will be understood by those skilled in the art that various changes in form and details may be made thereto without departing from the spirit and scope of the present disclosure as defined by the appended claims.

What is claimed is:

1. A peptide comprising one or more amino acid sequence selected from a group consisting of SEQ ID NO 1 and SEQ ID NO 2.

2. The peptide according to claim 1, which binds specifically to a graphitic material.

3. The peptide according to claim 2, wherein the graphitic material is selected from graphite, graphene, highly oriented pyrolytic graphite (HOPG), carbon nanotube and fullerene.

4. The peptide according to claim 1, which is derived from an M13 phage display p8 peptide library.

5. A method for screening the peptide of claim 1, comprising:
(1) preparing an M13 phage display p8 peptide library;
(2) conjugating the M13 phage display p8 peptide library onto a surface of a graphitic material; and
(3) screening a peptide that specifically bind to the surface of the graphitic material from the M13 phage display p8 peptide library by removing peptides that are not bound to the surface of the graphitic material.

6. The method for screening the peptide according to claim 5, wherein the M13 phage display p8 peptide library is prepared by a method comprising:
(a) preparing a mutant M13 phage vector through site-directed mutation of an M13 phage vector; and
(b) preparing an M13 phage display p8 peptide library from the mutant M13 phage vector using a restriction enzyme.

7. A graphitic material comprising a graphitic surface on which the peptide according to claim 1 is bound.

8. The graphitic material comprising a graphitic surface on which the peptide is bound according to claim 7, which is selected from graphite, graphene, highly oriented pyrolytic graphite (HOPG), carbon nanotube and fullerene.

9. An M13 phage wherein the peptide according to claim 1 is displayed on a coat protein.

10. The M13 phage according to claim 9, wherein the coat protein is selected from a group consisting of p3, p6, p7, p8 and p9.

11. The M13 phage according to claim 10, wherein the coat protein is p8.

12. A graphitic material comprising a graphitic surface on which the M13 phage according to claim 9 is arranged.

13. The graphitic material including a graphitic surface on which the M13 phage is arranged according to claim 12, which is selected from graphite, graphene, highly oriented pyrolytic graphite (HOPG), carbon nanotube and fullerene.

14. The graphitic material including a graphitic surface on which the M13 phage is arranged according to claim 12, wherein the M13 phage is arranged on the graphitic surface with directionality.

15. The graphitic material including a graphitic surface on which the M13 phage is arranged according to claim 12, wherein the M13 phage is arranged on the graphitic surface in a row.
